(19) United States
(12) Patent Application Publication
(10) Pub. No.: US 2010/0268301 A1
(43) Pub. Date: Oct. 21, 2010

(54) IMAGE PROCESSING ALGORITHM FOR CUEING SALIENT REGIONS

(75) Inventors: Neha J. Parikh, Los Angeles, CA (US); James D. Weiland, Los Angeles, CA (US); Mark S. Humayun, Los Angeles, CA (US)

Correspondence Address: Hogan Lovells US LLP, 1999 AVENUE OF THE STARS, SUITE 1400, LOS ANGELES, CA, 90067, US

(73) Assignee: University of Southern California, Los Angeles, CA (US)

(21) Appl. No.: 12/718,790

(22) Filed: Mar. 5, 2010

Related U.S. Application Data
(60) Provisional application No. 61/158,030, filed on Mar. 6, 2009.

(51) Int. Cl. A61N 1/05; G06K 9/46

(52) U.S. Cl. 607/54; 382/190; 382/165

(57) ABSTRACT

A method for cueing salient regions of an image in an image processing device is provided and includes the steps of extracting three information streams from the image. A set of Gaussian pyramids are formed from the three information streams by performing eight levels of decimation by a factor two. A set of feature maps are formed from a portion of the set of Gaussian pyramids. The set of feature maps are resized and summed to form a set of conspicuity maps. The set of conspicuity maps are normalized, weighted and summed to form the saliency map.

### CLAIM TO PRIORITY

This application claims priority to U.S. Provisional Application Ser. No. 61/158,030 filed on Mar. 6, 2009, the content of which is incorporated herein by reference.

### FUNDING

This invention was made with support in part by National Science Foundation grant EEC-0310723. Therefore, the U.S. government has certain rights.

### FIELD OF THE INVENTION

The present invention relates in general to an image processing method for cueing salient regions. More specifically, the invention provides an algorithm capable of detecting and cueing important objects in the scene and having low computational complexity so that it could be executable on a portable/wearable/implantable electronics module.

### DESCRIPTION OF THE RELATED ART

A visual attention based saliency detection model is described in Itti, L., Koch, C., & Niebur, E. (1998). “A model of saliency-based visual-attention for rapid scene analysis.” IEEE Transactions on Pattern Analysis and Machine Intelligence, 20, 1254-1259, which is incorporated herein by reference. Itti et al. is built upon the architecture proposed in Koch, C., & Ullman, S. (1985). “Shifts in selective visual attention: towards the underlying neural circuitry.” Human Neurobiology, 4, 219-227, which is incorporated herein by reference. Specifically, Koch et al. provides a primate model bottom-up model of visual processing. The model represents the pre-attentive processing in the primate visual system, in order to select the locations of interest which would be further analyzed by the complex processes in the attention stage. Three types of information—intensity, color and orientation are extracted from an image to form seven information streams—intensity, Red-Green opponent color, Blue-Yellow opponent, 0 degree orientation, 45 degree orientation, 90 degree orientation and 135 degree orientation. These seven streams of information undergo eight successive levels of decimation by a factor of two and low pass filtering to form Gaussian pyramids. Based on the center-surround mechanism, feature maps are created using the Gaussian image pyramids. Six feature maps are produced for every stream of information, for a total of forty-two feature maps for one processed image. Six feature maps correspond to intensity, twelve feature maps correspond to color and twenty four maps correspond to orientation. After iterative normalization to bring the different modalities at comparable levels, the feature-maps are combined into a saliency map from which salient regions are detected based on highest to lowest pixel gray scale levels. The saliency map represents the conspicuity, or saliency, at every location in a given image by a scalar quantity to present locations of importance. Itti, L., Koch, C. (2000), “A saliency-based search mechanism for overt and covert shifts of visual attention,” Vision Research, 40, 1489-1506, further describes a saliency based visual search and is also herein incorporated by reference.

### BRIEF SUMMARY OF THE INVENTION

The present invention provides an image processing method with low computational complexity for detecting salient regions in an image frame. The method is preferably implemented in a portable saliency cueing apparatus where the user's gaze is directed towards important objects in the peripheral visual field. The portable saliency cueing apparatus is further used with a retinal prosthesis. Such a system may aid implant recipients in understanding unknown environments by directing them to look towards important areas. The computational efficiency of the method advantageously increases the real-time performance of the image processing. The salient regions determined in the image are then communicated to the user through audio, visual or tactile cues. In this manner, the field of view is effectively increased. The originally proposed model of Koch et al. requires a much larger number of calculations that preclude it's practical use in a real-time, portable system.

Accordingly, one embodiment of the invention is a method for cueing salient regions of an image in an image processing device including the steps of extracting three information streams from the image. A set of Gaussian pyramids are formed from the three information streams by performing eight levels of decimation by a factor two. A set of feature maps are formed from a portion of the set of Gaussian pyramids. The set of feature maps are resized and summed to form a set of conspicuity maps. The set of conspicuity maps are normalized, weighted and summed to form the saliency map. The three information streams include saturation, intensity and high-pass information. The image is converted from a RGB color space to an HSI color space before the step of extracting. The feature maps are created from the pyramid levels 3, 4, 6 and 7 for each of the information streams. The set of conspicuity maps include intensity, color and Laplacian conspicuity maps. The intensity and color conspicuity maps are normalized with three iterations and the Laplacian conspicuity map is normalized with one iteration. The conspicuity maps of intensity, color and Laplacian undergo a simple averaging to form the saliency map. Alternatively, the conspicuity maps may be given weighting factors. A highest gray level pixel in the saliency map is a most salient region. An indication of the most salient region is cued to a user through an audio, visual or tactile cue.

In another embodiment of the present invention, an image processing program is embodied on a computer readable medium and includes the steps of extracting three information streams from the image. A set of Gaussian pyramids are formed from the three information streams by performing eight levels of decimation by a factor two. A set of feature maps are formed from a portion of the set of Gaussian pyramids. The set of feature maps are resized and summed to form a set of conspicuity maps. The set of conspicuity maps are normalized, weighted and summed to form the saliency map. The three information streams include saturation, intensity and high-pass information. The image is converted from a RGB color space to an HSI color space before the step of extracting. The feature maps are created from the pyramid levels 3, 4, 6 and 7 for each of the information streams. The set of conspicuity maps include intensity, color and Laplacian conspicuity maps. The intensity and the color conspicuity maps are normalized with three iterations and the Laplacian conspicuity map is normalized with one iteration. The conspicuity maps of intensity, color and Laplacian undergo a simple averaging to form the saliency map. A highest gray level pixel in the saliency map is a most salient region. An indication of the most salient region is cued to a user through an audio, visual or tactile cue.

In yet another embodiment of the present invention, a portable saliency cueing apparatus includes an image capture section capturing an image, a processor for calculating salient regions from the captured image, a storage section and a cueing section for cueing the salient regions. The processor extracts three information streams from the image provided by the image capture section, forms a set of Gaussian pyramids from the three information streams by performing eight levels of decimation by a factor two, and forms a set of feature maps from a portion of the set of Gaussian pyramids. The processor next resizes and sums the set of feature maps to form a set of conspicuity maps, which are then normalized, weighted and summed to form the saliency map. The storage section stores the saliency map, and the cueing section cues salient regions derived from the saliency map. The portable saliency cueing apparatus provides audio, visual or tactile cues to a user. The portable saliency cueing apparatus further includes a retinal prosthesis providing visual assistance for a blind user. The cueing section provides cues outside a field of view of the retinal prosthesis.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE DRAWINGS

FIG. 1 is a flowchart according to one embodiment of the invention.

FIG. 2A is a saliency map according to another embodiment of the invention.

FIG. 2B is a saliency map according to a prior art primate model.

FIG. 3 is a block diagram of a portable saliency cueing apparatus according to yet another embodiment of the invention.

### DETAILED DESCRIPTION OF THE INVENTION

The present invention is a method of detecting and cueing important objects in the scene and having low computational complexity. Preferably, the method is executed on a portable/wearable/implantable electronics module. The method is particularly useful in aiding implant recipients of retinal prosthesis in understanding unknown environments by directing them to look towards important areas. The invention is not limited to a retinal prosthesis, as the method is useful in video surveillance, automated inspection, digital image processing, video stabilization, automatic obstacle avoidance, and other assistive devices for blind. The inventive method is useful in any image processing application requiring detection of salient regions under processing and power constraints.

The present invention is loosely based on Itti's model of primate visual attention (hereinafter referred to as the primate model), with several crucial differences. First, the input image data is converted from the RGB color space into the Hue-Saturation-Intensity (HSI) color space to provide three information streams of saturation, intensity values and the high pass information of the image. Only three information streams are used in the present invention, versus seven in the primate model. Next, Gaussian pyramids are created at nine levels by successive decimation and low pass filtering but only the last two levels of the center and surround portions of the pyramids are used in constructing the feature maps. The center portions correspond to pyramid levels 1-4 and the surround portions are pyramid levels 5-8. The last levels of the center and surround pyramids signify the low pass information for the center and surround pyramids, such as when using feature maps (3-6), (3-7) and (4-7). The primate model utilizes all the created levels in constructing the feature maps. As discussed in further detail below, the feature maps undergo a normalization process and are combined to form a final saliency map from which salient regions are detected. Iterative normalization is implemented with one or three iterations compared to at least five iterations for the primate model. The present method thus concentrates more on low frequency which leads to the detection of larger details than small and fine details. In this manner, the computational complexity of the method is thus reduced over the primate model so as to allow execution on a portable processor for real-time applications.

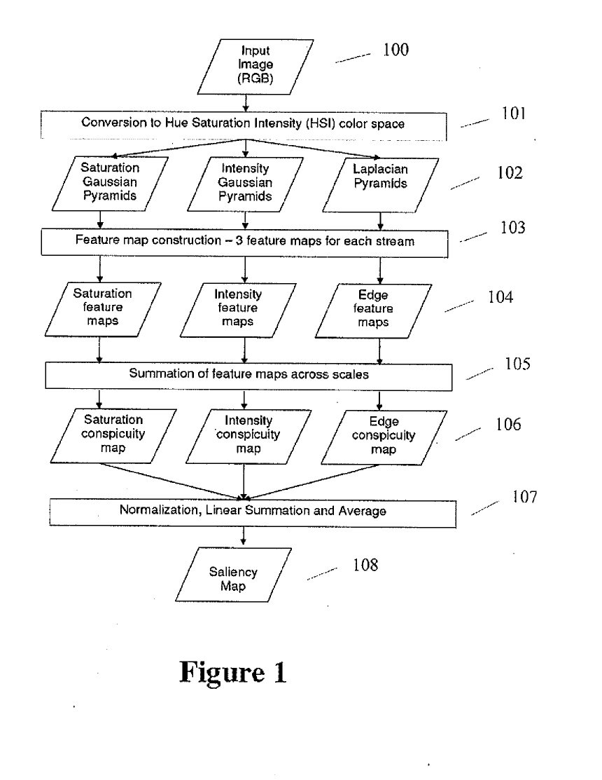

FIG. 1 is a flowchart of one embodiment of the invention. In step 100, input image data is provided in a format such as an RGB color space. If not already done so, the image data is converted in step 101 into the HSI color space. In step 102, the information streams of saturation, intensity values and high-pass information are extracted from the image data and are used to form dyadic Gaussian pyramids for the saturation and intensity information and Laplacian pyramids for the high-pass information. Specifically, each stream undergoes eight levels of successive decimation by a factor of two and low-pass filtering to form the Gaussian and Laplacian pyramids. Taking into consideration that the information streams of the original image lie at level 0, the Gaussian pyramids are a nine level pyramid scheme. Four levels of the Gaussian pyramids at levels 3, 4, 6 and 7 are used to create three feature-maps in step 103 using a center-surround mechanism for each of the information streams. The feature maps are obtained by a point-by-point subtraction of image matrices preferably at levels (3-6), (3-7) and (4-7) when the original image is level zero of the pyramid. Alternatively, the levels (4-8), (5-8) and (5-9) may be used. The image matrices of step 104 are resized to the finer scales before the subtraction of step 105. The result in step 106 are conspicuity maps for each of the respective streams. The feature maps are added for each of the streams to create the conspicuity maps of that particular information stream (step 106). The conspicuity maps thus obtained are resized to the size of the matrix at level 4. In step 107, the intensity and color conspicuity maps undergo a normalization process with three iterations (based on the iterative normalization process proposed by Itti et al.) and the Laplacian conspicuity map undergoes a one iteration normalization process. Normalization is an iterative process that promotes maps with a small number of peaks with strong activity and suppresses maps with many peaks of similar activity. The conspicuity maps of intensity, color and Laplacian undergo a simple averaging to form the saliency map of step 108. Alternatively, the maps are added with respective weighting factors of 1.5, 1 and 1.75 for the intensity, color and Laplacian conspicuity maps to form the final saliency map. In analyzing the saliency map, the region around the highest gray level pixel in the final saliency map is the most salient region. The second most salient region would be a region around the highest gray level pixel after masking out the most salient region and so on.

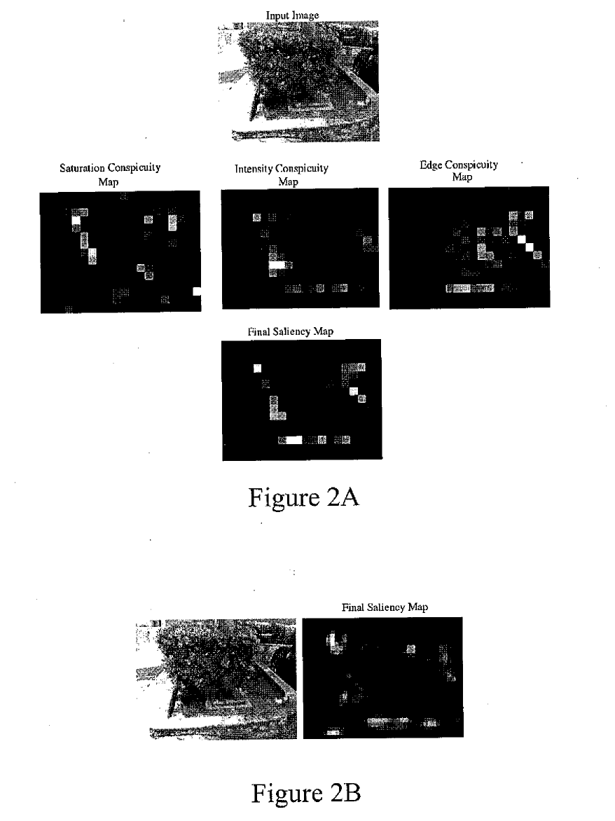

The salient map provided by the process is formed in a computationally efficient manner. Specifically, the present invention produces eighteen feature maps versus forty two for the primate model. Instead of using two color opponent streams as found in the primate retina, the present method uses color saturation. Color saturation information indicates purer hues with higher grayscale values and impure hues with lower grayscale values. Furthermore, only one stream of edge information (high pass information) is used instead of the four orientation streams in the primate model. Thus, the inventive method focuses on the coarser scales representing low spatial frequency information in the image. For example, FIG. 2A illustrates the input images and subsequent conspicuity maps and saliency map formed using the inventive method. FIG. 2B illustrates the saliency map using the primate model for the same image.

The present invention can be implemented on a digital signal processor such as the DSP TMS320DM642, 720 MHz Imaging Developers Kit, produced by Texas Instruments, Inc. Implementation of the image processing method on this DSP provides image processing at rates between 1-2 frames/sec. As a comparison, algorithms implementing just one of the seven information streams in the primate model run at less than 1 frame per second on the same hardware. The computational efficiency of the inventive method is crucial in implementing in a portable system where processing and energy are limited. An example of a specific implementation of the saliency method where speed and efficiency are important is provided below.

An electronic retinal prosthesis is known for treating blinding diseases such as retinitis pigmentosa (RP) and age-related macular degernation (AMD). In RP and AMD, the photoreceptor cells are affected while other retinal cells remain relatively intact. The retinal prosthesis aims to provide partial vision by electrically activating the remaining cells of the retina. Current implementations utilize external components to acquire and code image data for transmission to an implanted retinal stimulator. However, while human monocular vision has a field of view close to 160°, the retinal prosthesis stimulates only the central 15-20° field of view. Presently, continuous head scanning is required by the user of the retinal prosthesis to understand the important elements in the visual field, which is both time-consuming and inefficient. Therefore, there is a need to overcome the loss of peripheral information due to the limited field of view.

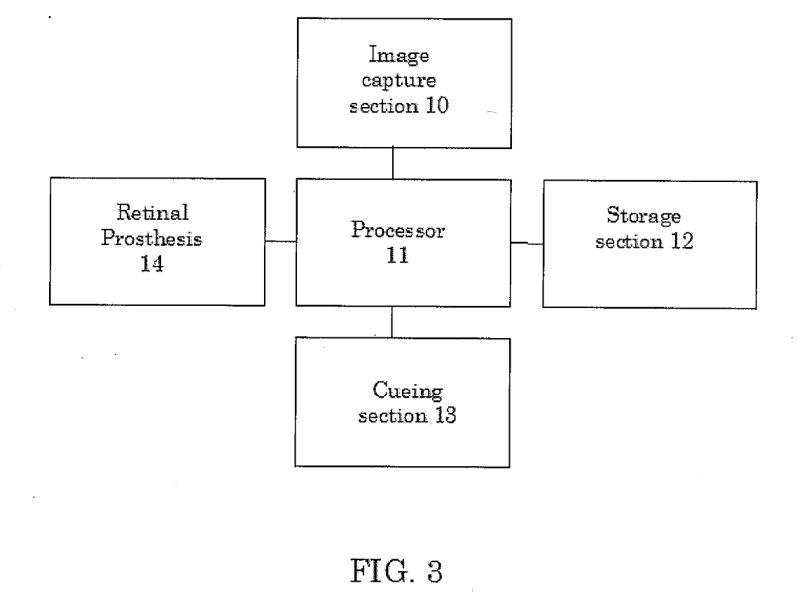

The above described image processing method for detecting salient regions in an image frame is preferably implemented in a portable saliency cueing apparatus for use in conjunction with a retinal prosthesis, to identify and cue users to important objects in a peripheral region outside the scope of the retinal prosthesis. As shown in FIG. 3, a saliency cueing system includes a processor 11, such as a DSP, for calculating the salient regions. An image capture section 10 is provided for capturing an image to be processed. A storage section 12 stores images and saliency maps and a cueing section 13 provides cues to a user. When the saliency method is implemented in conjunction with a retinal prosthesis, the user may be given one or more cues in the decreasing order of saliency by the cueing section 13. Once given a cue, the user can then scan the region around the direction of the cue(s) instead of scanning the entire scene which can be more time consuming. The method and apparatus can map salient regions to eight predetermined regions (regions to the left, right, top, down, top-left, top-right, bottom-left and bottom-right) falling outside the field of view. The cue can, for example, be emitted from an audio device providing feedback indicating the relative position of the salient region or from a predetermined sound emanating from the direction of the salient region. Upon hearing the audio cue, the user will know to direct their gaze to shift their field of view towards the detected salient region. The cue can also be provided visually through the retinal prosthesis or some other means with visual symbols indicating the direction of the salient region. In another embodiment, tactile feedback can be provided to a user to provide an indication of the location of the salient region. For example, a user who feels a vibration at a predetermined location such as their left hand will understand this to be the cue to turn their head to the left to visualize the detected salient region. Three to five saliency cues may be generated per image from the algorithm. It is important to note that the application of the primate model to a portable system such as the retinal prosthesis is impractical given the time-consuming calculations required. Furthermore, for obstacle avoidance and route planning, visually impaired individuals are likely to be more interested in large objects in their path rather than in the small details. In such a case, the inventive saliency method is advantageous. Moreover, the use of a computationally efficient cueing method reduces the power consumption of a portable processor to allow portable use the retinal prosthesis system that may rely on battery power.

While the invention has been described with respect to certain specified embodiments and applications, those skilled in the art will appreciate other variations, embodiments and applications of the invention not explicitly described. This application covers those variations, methods and applications that would be apparent to those of ordinary skill in the art.

1. A method for cueing salient regions of an image in an image processing device, comprising the steps of:
extracting three information streams from the image;
forming a set of Gaussian pyramids from the three information streams by performing eight levels of decimation by a factor two;
forming a set of feature maps from a portion of the set of Gaussian pyramids;
resizing and summing the set of feature maps to form a set of conspicuity maps;
normalizing, weighting and summing the set of conspicuity maps to form the saliency map.

2. The method of claim 1, wherein the three information streams include saturation, intensity and high-pass information.

3. The method of claim 1, further comprising the steps of:
converting the image from an Red-Green-Blue (RGB) color space to a Hue-Saturation-Intensity (HSI) color space before the step of extracting.

4. The method of claim 1, wherein the feature maps are created from the pyramid levels 3, 4, 6 and 7 for each of the information streams.

5. The method of claim 1, wherein the set of conspicuity maps include intensity, color and Laplacian conspicuity maps;
further comprising the steps of normalizing the intensity and the color conspicuity maps with three iterations and normalizing the Laplacian conspicuity map with one iteration.

6. The method of claim 5, wherein the conspicuity maps of intensity, color and Laplacian undergo a simple averaging to form the saliency map.

7. The method of claim 1, wherein a highest gray level pixel in the saliency map is a most salient region.

8. The method of claim 7, further comprising the steps of:
cueing an indication of the most salient region to a user through an audio, visual or tactile cues.

9. A computer readable medium encoded with an image processing program for cueing salient regions, comprising the steps of:
extracting three information streams from the image;
forming a set of Gaussian pyramids from the three information streams by performing eight levels of decimation by a factor two;
forming a set of feature maps from a portion of the set of Gaussian pyramids;
resizing and summing the set of feature maps to form a set of conspicuity maps;
normalizing, weighting and summing the set of conspicuity maps to form the saliency map.

10. The computer readable medium of claim 9, wherein the three information streams include saturation, intensity and high-pass information.

11. The computer readable medium of claim 9, further comprising the steps of:
converting the image from an Red-Green-Blue (RGB) color space to the Hue-Saturation-Intensity (HSI) color space before the step of extracting.

12. The computer readable medium of claim 9, wherein the feature maps are created from the pyramid levels 3, 4, 6 and 7 for each of the information streams.

13. The computer readable medium of claim 9, wherein the set of conspicuity maps include intensity, color and Laplacian conspicuity maps;
further comprising the steps of normalizing intensity and color conspicuity maps with three iterations and normalizing a Laplacian conspicuity map with one iteration.

14. The computer readable medium of claim 13, wherein the conspicuity maps of intensity, color and Laplacian undergo a simple averaging to form the saliency map.

15. The computer readable medium of claim 9, wherein a highest gray level pixel in the saliency map is a most salient region.

16. The computer readable medium of claim 15, further comprising the steps of:
cueing an indication of the most salient region to a user through an audio, visual or tactile cue.

17. A portable saliency cueing apparatus comprising:
an image capture section capturing an image; and
a processor for calculating salient regions from the captured image;
a storage section;
a cueing section for cueing the salient regions;
wherein the processor extracts three information streams from the image provided by the image capture device, the processor forms a set of Gaussian pyramids from the three information streams by performing eight levels of decimation by a factor two, the processor forms a set of feature maps from a portion of the set of Gaussian pyramids, the processor resizes and sums the set of feature maps to form a set of conspicuity maps, the processor normalizes, weights and sums the set of conspicuity maps to form the saliency map;
wherein the storage section stores the saliency map,
wherein the cueing section cues salient regions derived from the saliency map.

18. The portable saliency cueing apparatus of claim 17, wherein the cueing section provides audio, visual or tactile cues to a user.

19. The portable saliency cueing apparatus of claim 17, further comprising:
a retinal prosthesis providing visual assistance for a blind user.

20. The portable saliency cueing apparatus of claim 19, wherein the cueing section provides cues outside of a field of view of the retinal prosthesis.
